(19) United States
(12) Patent Application Publication
(10) Pub. No.: US 2026/0183311 A1
(43) Pub. Date: Jul. 2, 2026

(54) COMPOUNDS DERIVED FROM STEROLS FOR THE TREATMENT OF A DISEASE ASSOCIATED WITH A MITOCHONDRIAL DEFICIT

(71) Applicant: DENDROGENIX, Liège (BE)

(72) Inventors: Stéphane SILVENTE, Liège (BE); Nicolas CARON, Clavier (BE); Arnaud RIVES, Tilff (BE); Quentin MARLIER, Emines (BE); Hélène MICHAUX, Liège (BE); Dario MOSCA, Namur (BE); Maxime GILSOUL, Ougrée (BE)

(73) Assignee: DENDROGENIX, Liège (BE)

(21) Appl. No.: 18/875,115

(22) PCT Filed: Jun. 14, 2023
(86) PCT No.: PCT/EP2023/065981

§ 371 Date: Dec. 13, 2024

(30) Foreign Application Priority Data
Jun. 14, 2022 (BE) BE2022/5469

(51) Int. Cl. A61K 31/575; A61K 9/08; A61K 31/58

(52) U.S. Cl. CPC A61K 31/575; A61K 9/08; A61K 31/58

(57) ABSTRACT

A sterol-based compound of formula (I) for its use in preventing, ameliorating and/or treating a mitochondrial deficiency-related pathology chosen from the group consisting of autism, spinal cord injury, multiple sclerosis, epilepsy, migraine, alcohol-related mental pathologies, ataxia, neuropathies, smoking-related cerebral and pulmonary disorders, MERRF syndrome and NARP syndrome. A pharmaceutical composition comprising at least one compound of formula (I).

### FIELD OF THE INVENTION

The invention relates to the field of mitochondrial deficiency-related pathologies. More precisely, the invention relates to sterol-based compounds of formula (I) and to a pharmaceutical composition comprising sterol-based compounds of formula (I) or a pharmaceutically acceptable salt of such a compound, for use in preventing, ameliorating and/or treating a mitochondrial deficiency-related pathology chosen from the group formed by autism, spinal cord injury, multiple sclerosis, epilepsy, migraine, alcohol-related mental pathologies, ataxia, neuropathies, smoking-related cerebral and pulmonary disorders, MERRF syndrome and NARP syndrome.

### BACKGROUND OF THE INVENTION

The mitochondrion is a fundamental organelle present in most eukaryotic cells. Its main role is to provide cells with the energy they need to ensure their survival and the functions they are expected to perform, by converting oxygen and food into energy sources such as adenosine triphosphate (ATP). Given this fundamental role played by the mitochondrion within the cell, a multitude of pathologies are associated with mitochondrial dysfunction, mainly affecting systems that require large amounts of energy, such as the muscles, the brain or the liver.

A defective or damaged mitochondrion will produce energy in a defective manner too. There are what are known as primary (inherited) and secondary (acquired) mitochondrial diseases. In both cases, the mitochondria no longer function correctly and will give rise to various pathologies. These pathologies have a connection with mitochondrial activity, mitochondrial dynamics (biogenesis, fusion, fission) and axonal transport. Mitochondrial diseases, or mitochondropathies, combine a disparate range of diseases relating to disorders of the mitochondrial respiratory chain.

There is a multitude of pathologies in which defects in mitochondrial activity and/or mitochondrial dynamics and/or axonal transport are observed, notably autism, spinal cord injury, multiple sclerosis, epilepsy and even migraine (Cleveland Clinic, Mitochondrial diseases, https://my.clevelandclinic.org/health/diseases/15612-mitochondrial-diseases).

Aside from natural means such as exercising or adopting a healthy lifestyle (avoidance of alcohol, sleep deprivation, drastic diets, etc.), there are currently very few molecules that are capable of improving mitochondrial function in general. Coenzyme Q10, vitamin B (thiamine, riboflavin), alpha-lipoic acid, L-carnitine, creatine, L-arginine, idebenone, KH176, Elamipretide, Bezafibrate, resveratrol, omaveloxolone, rapamycin (patent U.S. Pat. No. 10,792,287), and Nicotinamide Adenine Dinucleotide (NAD+) precursors are regularly mentioned as potential treatments for mitochondrial diseases, but none of them has passed through all the clinical phases. Only vitamin B12 can be administered at present (Treatment for mitochondrial diseases, Tongling Liufu et al., Sep. 9, 2020, https://doi.org/10.1515/revneuro-2020-0034; Molecular Genetics and Metabolism, vol. 131, September-October 2020, pages 1-13, Clinical trials in mitochondrial disorders, an update, Mohammed Almannai et al., doi: 10.1016/Avmqme.2020.10.002).

There is thus currently no FDA-approved therapy targeting the treatment of mitochondrial dysfunction (Trends in Molecular Medicine, Special issue: Mitochondria—from diagnosis to treatment review, vol. 26, issue 1, pages 40-57, Jan. 1, 2020, https://doi.org/10.1016/j.molmed.2019.09.002).

Mitochondropathies are predominantly the result of mutations or deletions in genes encoding mitochondrial proteins. Mitochondrial function may thus be impaired either by genetic mutation or by acquired or iatrogenic mitochondrial damage. Improving or restoring mitochondrial function when it is deficient is therefore a fundamental challenge in the fight against all these pathologies, or at least against some of those identified as being the consequence of mitochondrial deficiency.

The spectrum of autistic disorders is a neurodevelopmental disease leading to defects in social behavior and also cognitive disorders. Mitochondrial dysfunction is currently under consideration as a possible cause of this disorder, notably defects in mitochondrial dynamics (mitochondrial fission), mitochondrial biogenesis or axonal transport of these mitochondria (Seminar in Pediatric Neurology, vol. 25, October 2020, Richard E. Frye et al., Mitochondrial dysfunction in autism spectrum disorder: unique abnormalities and targeted treatments, https://doi.org/10.1016/j.spen.2020.100829).

During spinal cord injury, the axons degenerate. There is increasing evidence that mitochondria play a fundamental role in the degeneration/regeneration processes of these axons, whether in terms of mitochondrial activity, dynamics or mitochondrial axonal transport (Frontiers in aging neuroscience, Mar. 8, 2021, Biyao Wang et al., Mitochondrial behavior in axon degeneration and regeneration, https://doi.org/10.3389/fnagi.2021.650038).

Multiple sclerosis is a disease in which nerve demyelination leads to axonal damage and various neurological symptoms. Defects in mitochondrial activity and transport have also been observed in this pathology (Immunology and Inflammation, Neuroscience, Feb. 10, 2021, Sina C Rosenkranz et al., Enhancing mitochondrial activity in neurons protects against neurodegeneration in a mouse model of multiple sclerosis, https://elifesciences.org/articles/61798).

Mitochondrial dysfunction has also been identified as a potential cause of epilepsy (European Journal of Pediatric Neurology, vol. 24, pages 47-52, Jan. 1, 2020, Albert Lim et al., The mitochondrial epilepsies, https://www.ejpn-journal.com/article/S1090-3798(19)30441-6/fulltext). Antioxidants targeting mitochondrial oxidative stress have also been shown to be promising neuroprotective agents in epilepsy (Oxidative Medicine and Cellular Longevity, vol. 2020, Article ID 6687185, Nan Yang et al., Antioxidants targeting mitochondrial oxidative stress: promising neuroprotectants for epilepsy, https://doi.org/10.1155/2020/6687185). Repeated migraine attacks are regularly associated with metabolic changes in specific brain regions. These metabolic changes result from defects in mitochondrial function that are present in these regions (Headache: The Journal of Head and Face Pain, 2018, vol. 58: pages 45-52, Kraya T. et al., Prevalence of headache in patients with mitochondrial disease: a cross-sectional study, https://americanheadachesociety.org/news/journal-headache-mitochondrial-disease/).

Mitochondrial dysfunction has been described in alcohol-related mental pathologies. Several mental pathologies may be associated with alcohol consumption, such as independent major depression, bipolar disorder, anxiety or personality disorders (Shivani, R., Goldsmith, R. J., & Anthenelli, R. M. (2002). Alcoholism and Psychiatric Disorders: Diagnostic Challenges. Alcohol Research & Health, 26(2), 90-98). A mechanism has been described for ethanol toxicity in brain neurons, in which mitochondria are the main mediators and mitochondrial alterations correlate with the severity of ethanol consumption. Thus, improving the health of brain cell mitochondria is under consideration as a potential therapeutic target for treating ethanol-related pathologies (Ethanol Consumption Affects Neuronal Function: Role of the Mitochondria, Cheril Tapia-Rojas et al., Dec. 20, 2017, DOI: 10.5772/intechopen.71611). It has also been shown that adolescent alcohol consumption has a significant impact on mitochondrial bioenergetics during adulthood, and that this is not a transient change until consumption ceases, as previously thought (Neuroscience, vol. 406, May 15, 2019, pages 356-368, Cheril Tapia-Rojas et al., Adolescence binge alcohol consumption induces hippocampal mitochondrial impairment that persists during the adulthood).

Ataxia is a neuromuscular disease consisting of a lack of fine coordination of voluntary movements. It is linked to damage to the nervous system. Among the ataxias associated with mitochondrial deficiency are Friedreich's syndrome (Molecular and Cellular Neuroscience, vol. 102, January (2020), Anna Stepanova et al., Mitochondrial dysfunction in neurons in Friedreich's ataxia, https://www.sciencedirect.com/science/article/abs/pii/S1044743119301964?via %3Dihub), telangiectasias (Scientific Report 9, 4782 (2019), Blignaut, M. et al., Ataxia-Telangiectasia Mutated is located in cardiac mitochondria and impacts oxidative phosphorylation, https://www.nature.com/articles/s41598-019-41108-1); spinocerebellar ataxia (Cell Reports, vol. 26, issue 5, pages 1189-1202, Jan. 29, 2019, Metabolic and organelle morphology defects in mice and human patients define spinocerebellar ataxia type 7 as a mitochondrial disease, https://www.cell.com/cell-reports/fulltext/S22111247(19)300373?_returnURL=https %3A %2F %2Flinkinghub.elsevi er.com %2Fretrieve %2Fpii %2FS2211124719300373%3Fshowall %3Dtrue #relatedArticle s).

Neuropathies and myopathies have also been shown to have a connection with mitochondrial dysfunction (Mitochondrion, 56 (2021), 52-61, Jian-Qiang Lu et al., Mitochondrial neuropathy and neurogenic features in mitochondrial myopath., https://doi.org/10.1016/j.mito.2020.11.005).

Smoking-related cerebral and pulmonary disorders have been demonstrated in numerous publications, with nicotine or tobacco smoke having an impact on various mitochondrial activities. Mitochondria have been presented as a possible target of the action of nicotine in the brain (Journal of Bioenergetics and Biomembranes, 51, 259-276 (2019), Dominika Malinska et al., Mitochondria as a possible target for nicotine action) and disturbances in the mitochondrial function and structure of lung epithelial cells have been demonstrated by the action of cigarette smoke (American Journal of Physiology, vol. 318, No. 1, Jan. 7, 2020, Mathyar Aghapour et al., Mitochondria: at the crossroads of regulating lung epithelial cell function in chronic obstructive pulmonary disease, https://doi.org/10.1152/ajplung.00329.2019).

Finally, MERRF (Myoclonic Epilepsy with Ragged Red Fibers) syndrome and NARP (Neuropathy, Ataxia and Retinitis Pigmentosum) syndrome are both related to mitochondrial deficiency (Elsevier, Biochimica et Biphysica Acta (BBA) Molecular Basis of Disease, vol. 1866, issue 6, Jun. 1, 2020, Marina Vilanueva-Paz et al., Parkin-mediated mitophagy and autophagy flux disruption in cellular models of MERRF syndrome; Elsevier, The International Journal of Biochemistry & Cell Biology, vol. 45, issue A, January 2013, pages 141-150, Magdanela Lebiedzinska et al., Disrupted ATP synthase activity and mitochondrial hyperpolarization-dependent oxidative stress is associated with p66Shc phosphorylation in fibroblasts of NAR patients).

U.S. Pat. No. 10,792,287 describes the use of pharmaceutical compounds (zolpidene with the addition of rapamycin or idebedone) for their beneficial effect on mitochondrial ATP production, inhibition of inflammation and for treating various mitochondrial pathologies such as vision loss and Leber's hereditary optic neuropathy.

U.S. Pat. No. 10,272,056 also describes the use of butyrate-type compounds for their beneficial effects on mitochondrial biogenesis, the increase in mitochondrial mass, the increase in ATP production and their efficacy in several pathologies such as neurodegenerative diseases, cardiovascular, neurometabolic, muscular, renal and metabolic diseases, and certain particular syndromes such as MELAS (Mitochondrial Encephalopathy with Lactic Acidosis and Stroke-like episodes) and MERRF syndrome.

### SUMMARY OF THE INVENTION

The Applicant has found, surprisingly, that a sterol-based compound of formula (I), or a pharmaceutically acceptable salt of such a compound, is useful in preventing, ameliorating and/or treating a mitochondrial deficiency-related pathology caused by a deficiency in mitochondrial dynamics and/or transport and/or activity. More precisely, the Applicant has found that a sterol-based compound of formula (I) or a pharmaceutically acceptable salt of such a compound, is useful in preventing, ameliorating and/or treating a mitochondrial deficiency-related pathology chosen from the group consisting of autism, spinal cord injury, multiple sclerosis, epilepsy, migraine, alcohol-related mental pathologies, ataxia, neuropathies, smoking-related cerebral and pulmonary disorders, MERRF syndrome and NARP syndrome.

The object of the invention is to provide sterol-based compounds and a pharmaceutical composition comprising them for their use in preventing, ameliorating and/or treating a mitochondrial deficiency-related disease chosen from the group consisting of autism, spinal cord injury, multiple sclerosis, epilepsy, migraine, alcohol-related mental pathologies, ataxia, neuropathies, smoking-related cerebral and pulmonary disorders, MERRF syndrome and NARP syndrome.

To do this, the invention provides a compound of formula (I):

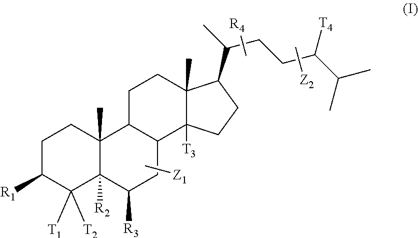

in which
R₁=OH, F, OCₙH₂ₙ₊₁, OC(O)R, OC(O)OR, OC(O)NHR or OP(O)(OR)₂ with R=H or CₙH₂ₙ₊₁, with 1≤n≤8,
R₂=H or OH;
R₃=−NR₅R₆,
R₅ being H or −(CH₂)₃NH₂, and
R₆ being included in the group formed by —(CH₂)₃NR₇(CH₂)₄NHR₇, —(CH₂)₃NHR₇, —(CH₂)₄NHR₇, —(CH₂)₄N R₇(CH₂)₃NHR₇, —(CH₂)₃N R₇(CH₂)₄N R₇(CH₂)₃NHR₇, —(CH₂)₂-imidazol-4-yl, —(CH₂)₂-indol-3-yl, with R₇=H, C(O)OCH₃ or C(O)OC(CH₃)₃; and
R₄=H or OH in position 20, 22, 24, 25, 26 or 27, positioned to create an asymmetric center of R or S configuration;
Z₁ and Z₂ each represent the number of double bonds between carbon atoms C7 and C8 and C22 and C23 respectively (either 0 or 1); T₁, T₂ and T₃=H or CH₃ independently of each other; T₄=H, CH₃, C₂H₅ positioned to obtain an asymmetric center of R or S configuration in position 24;
for its use in the treatment of a mitochondrial deficiency-related pathology chosen from the group consisting of autism, spinal cord injury, multiple sclerosis, epilepsy, migraine, alcohol-related mental pathologies, ataxia, neuropathies, smoking-related cerebral and pulmonary disorders, MERRF syndrome and NARP syndrome.

The mention of (O) in the definition of a radical according to formula (I) means that the oxygen is bonded by two bonds.

The compound of formula (I) is defined by: Z₁=Z₂=0; R₁=R₂=OH; R₄=H; R₅=H; R₆=—(CH₂)₃−NC(O)OC(CH₃)₃—(CH₂)₄—NHC(O)OC(CH₃)₃; T₁=T₂=T₃=T₄=H is called DX243BOC or DXboc and is illustrated in table 2.

The substituent or radical C(O)OC(CH₃)₃ is also known as a tert-butoxycarbonyl or Boc functional group.

The compound of formula (I) belongs to the steroid group. The numbering of the carbon atoms of the compound of formula (I) thus follows the nomenclature defined by IUPAC in Pure & Appl. Chem., Vol. 61, No. 10, pages 1783-1822, 1989. The numbering of the carbon atoms of a compound belonging to the steroid group according to IUPAC is illustrated below:

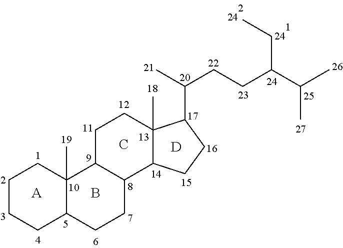

The methods for preparing the compound of formula (I) have already been described previously, notably in de Medina, P. et al. Synthesis of New, the Treatment of Cancer and Neurodegenerative Diseases. Journal of Medicinal Chemistry, 52(23), 2009, pp. 7765-7777.

In addition, the compound may have one or more of the following features, considered singly or in combination. According to one embodiment, the compound of formula (I) is defined by R₁=OH, F, OCₙH₂ₙ₊₁, OC(O)R, OC(O)OR, OC(O)NHR or OP(O)(OR)₂ with R=H or CₙH₂ₙ₊₁, with 1≤n≤8;
R₂=OH;
R₃═—NR₅R₆;
R₅=H;
R₆=—(CH₂)₃NR₇(CH₂)₄NHR₇, —(CH₂)₃NHR₇, —(CH₂)₄NHR₇, —(CH₂)₄N R₇(CH₂)₃NHR₇, —(CH₂)₃N R₇(CH₂)₄N R₇(CH₂)₃NHR₇, —(CH₂)₂-imidazol-4-yl, —(CH₂)₂-indol-3-yl, with R₇=H, C(O)OCH₃ or C(O)OC(CH₃)₃;
Z₁=0 or 1;
Z₂=0;
R₄=H.

According to one embodiment, the compound of formula (I) is more precisely defined by Z₁=0, Z₂=0; R₁=R₂=OH; R₄=H; R₅=H; T₁=T₂=T₃=T₄=H, with the other radicals R₃, R₆ and R₇ as defined previously.

According to one embodiment, the compound of formula (I) is defined more precisely by Z₁=0; Z₂=0; R₁=R₂=OH; R₄=H; R₅=H; T₁=T₂=T₃=T₄=H, with R₆=—(CH₂)₄NH(CH₂)₃NHR₇ and R₇=C(O)CH₃. This compound named DX249 is 5α-hydroxy-6β-[3-(4-aminobutylacetamide)propylamino]cholestan-3β-ol.

According to one embodiment, the compound of formula (I) is more precisely defined by Z₁=0; Z₂=0; R₁=R₂=OH; R₄=H; R₅=H; T₁=T₂=T₃=T₄=H, with R₆=—(CH₂)₂-imidazol-4-yl. This compound named DX101 is 5α-hydroxy-6β-[2-(1H-imidazol-4-yl)ethylamino]cholestan-3β-ol.

According to one embodiment, the compound of formula (I) is more precisely defined by Z₁=0; Z₂=0; R₁=R₂=OH; R₄=H; R₅=H; T₁=T₂=T₃=T₄=H, with R₆=—(CH₂)₃N R₇(CH₂)₄NHR₇, —(CH₂)₄NR₇(CH₂)₃NHR₇, —(CH₂)₃NR₇(CH₂)₄NH(CH₂)₃NHR₇; or —(CH₂)₄NHR₇; and R₇=H. These compounds respectively named DX243, DX245, DX301 and DX401 are:
5α-hydroxy-6β-[3-(4-aminobutylamino)propylamino]cholestan-3β-ol (DX243)
5α-hydroxy-6β-[4-(3-aminobutylamino)propylamino]cholestan-3β-ol (DX245)
5α-hydroxy-6β-{3-[4-(3-aminopropylamino)butylamino]propylamino}cholestan-3β-ol (DX301)
5α-hydroxy-6β-(4-aminobutylamino)cholestan-3β-ol (DX401).

According to one embodiment, the compound of formula (I) is defined more precisely by Z₁=0, Z₂₌₀; R₁=R₂=OH; R₄=H; R₅=H; T₁=T₂=T₃=T₄=H, with R₆=—(CH₂)₃NR₇(CH₂)₄NHR₇ and R₇=C(O)OC(CH₃)₃. This compound named DX243BOC is 5α-hydroxy-6β-[3-(4-tert-butyloxycarbonylaminobutyl-tert-butyloxycarbonylamino)propylamino]cholestan-3β-ol.

According to one embodiment, the compound of formula (I) is more precisely defined by Z₁=1, Z₂=0; R₁=R₂=OH; R₄=H; R₅=H; T₁=T₂=T₃=T₄=H, with the other radicals R₃, R₆ and R₇ as defined previously.

According to one embodiment, the compound of formula (I) is more precisely defined by Z₁=1; Z₂=0; R₁=R₂=OH; R₄=H; R₅=H; T₁=T₂=T₃=T₄=H, with R₆=—(CH₂)₃NH(CH₂)₄NHR₇; —(CH₂)₄NH(CH₂)₃NHR₇; or —(CH₂)₃NH(CH₂)₄NH(CH₂)₃NHR₇; and R₇=H. These compounds respectively named DX242, DX244 and DX302 are:
5α-hydroxy-6β3-[3-(4-aminobutylamino)propylamino]cholest-7-en-3β-ol (DX242)
5α-hydroxy-6β3-[4-(3-aminobutylamino)propylamino]cholest-7-en-3β-ol (DX244)
5α-hydroxy-6β-{3-[4-(3-aminopropylamino)butylamino]propylamino}cholest-7-en-3ββ-ol (DX302).

According to one embodiment, the mitochondrial deficiency-related pathology is due either to a deficiency in mitochondrial dynamics and/or transport and/or activity. Table 1 illustrates the specificity of the deficiencies according to the pathology.

TABLE 1

| | | | Clinical | Mitochondrion | | |
|---|---|---|---|---|---|---|
| | Prevalence | | trial | Dynamics | | |
| | (2020) | | (2021) | (fusion/fission) | Transport | Activity |
| Neuropathies | 300 million | | 2523 | ✓ | ✓ | ✓ |
| | (2015) | | | | | |
| Spinal cord injury | 3.5 | million | 1377 | ✓ | ✓ | ✓ |
| Autism spectrum | 12 | million | 1172 | ✓ | ✓ | ✓ |
| disorder | | | | | | |
| Ataxia | 1 case per | | 299 | ✓ | ✓ | ✓ |
| | 10 000 | | | | | |
| | (2014) | | | | | |
| Alcohol-related | 99.2 million | | 679 | ✓ | ✓ | ✓ |
| mental | (2016) | | | | | |
| pathologies | | | | | | |
| (AUD) | | | | | | |
| Multiple sclerosis | 3 | million | 2452 | | ✓ | ✓ |
| Migraine | 840 | million | 1143 | ✓ | | ✓ |
| Epilepsy | 50 | million | 1623 | ✓ | | |
| Smoking-related | 1.3 | billion | 518 | ✓ | | ✓ |

| | | | | |
|---|---|---|---|---|
| mitochondrial | | | | |
| disorders | | | | |
| MERRF | <1 case per | 1 | ✓ | ✓ |
| syndrome | 100 000 | | | |
| NARP syndrome | <1 case per | 2 | | ✓ |
| | 10 000 | | | |

For the compound of formula (I) according to the invention, the mitochondrial deficiency-related pathology is due to a deficiency in mitochondrial dynamics and/or mitochondrial transport and/or mitochondrial activity.

In one embodiment, the pathologies associated with a deficiency in mitochondrial dynamics and mitochondrial transport and activity are neuropathies, spinal cord injury, autism, ataxia and alcohol-related mental pathologies.

In another embodiment, the pathologies associated with a deficiency in mitochondrial dynamics and mitochondrial activity are migraine, smoking-related cerebral and pulmonary disorders and MERRF syndrome.

In another embodiment, the pathology associated with a deficiency in mitochondrial transport and activity is multiple sclerosis.

In yet another embodiment, the pathology associated with a deficiency in mitochondrial activity is NARP syndrome.

A second subject according to the invention is a pharmaceutical composition comprising at least one compound of formula (I) and/or at least one pharmaceutically acceptable salt of at least one compound of formula (I), for its use in treating a mitochondrial deficiency-related pathology chosen from the group consisting of autism, spinal cord injury, multiple sclerosis, epilepsy, migraine, alcohol-related mental pathologies, ataxia, neuropathies, smoking-related cerebral and pulmonary disorders, MERRF syndrome and NARP syndrome.

The composition for use according to the invention may be administered in various forms suitable for the pathology to be treated. Thus, the various forms of administration comprise oral, topical, systemic, intravenous, subcutaneous, intraperitoneal, intramuscular, transdermal or transmucosal administration.

The composition according to the invention may be in any of the presentation forms normally used, depending on whether the composition is to be ingested, injected or applied to the skin or mucous membranes.

The composition according to the invention may comprise ingredients commonly used in this type of formulation, such as binders, flavoring agents, preserving agents or colorants, and may, in the case of food supplements or medicaments, be in tablet, granule or gel capsule form. Formulations according to the invention may also be in the form of food products such as beverages, or else in the form of suspensions or syrups.

According to a first variant, the various preparations are suitable for topical administration and include creams, oil-in-water and water-in-oil emulsions, milks, ointments, lotions, oils, balms, aqueous or aqueous-alcoholic or glycolic solutions, sera, powders, patches, sprays or any other product for external application, for instance medical devices or aerosol products also containing a pressurized propellant.

According to a second variant, the various compositions are suitable for injection; the composition may be in the form of an aqueous or oily lotion, or in the form of a serum.

According to a third variant, the various compositions are suitable for ingestion; the composition may be in the form of capsules, syrups, granules or tablets.

According to a preferential embodiment, the compositions according to the invention are more particularly intended for topical administration. These compositions must thus contain a dermatologically acceptable medium, i.e. a medium that is compatible with the skin and mucous membranes and cover all dermatological forms. These compositions may notably be in the form of creams, oil-in-water or water-in-oil emulsions or multiple emulsions, sera, solutions, suspensions, gels, milks, lotions, sticks or powders, and may be suitable for application to the skin and mucous membranes. These compositions comprise the excipients that are necessary for their formulation, such as solvents, emollients, thickeners, diluents, surfactants, antioxidants, bioactive agents, dyes, preserving agents and fragrances.

The compositions according to the invention also comprise any additive commonly used in the envisioned field of application and also the adjuvants required for their formulation, such as solvents, thickeners, diluents, antioxidants, dyes, sunscreens, self-tanning agents, pigments, fillers, preserving agents, fragrances, odor absorbers, dermatological or pharmaceutical active agents, essential oils, vitamins, essential fatty acids, surfactants, film-forming polymers, etc.

In any case, a person skilled in the art will take care to ensure that these adjuvants and the proportions thereof are chosen such that the desired advantageous properties of the compositions according to the invention are not adversely affected.

According to one embodiment, the composition for use according to the invention is in the form of an aqueous solution and has a concentration of compound of formula (I) of between 1 pmol·L⁻¹ and 1 mmol·L⁻¹, preferentially between 10 pmol·L⁻¹ and 0.1 mmol·L⁻¹, more preferentially between 0.1 nmol·L⁻¹ and 1 pmol·L⁻¹.

### BRIEF DESCRIPTION OF THE DRAWINGS

The invention will be better understood, and other aims, details, features and advantages thereof will appear more clearly from the following description of several particular embodiments of the invention, given purely as nonlimiting illustrations, with reference to the attached drawings.

FIG. 1 illustrates the results of a test using compounds of formula (I) on the activity of cortical neurons by measuring the optical density of the tetrazolium salt MTT (3-(4,5-dimethylthiazol-2-yl)-2,5-diphenyltetrazolium bromide). NT means not treated (untreated). The results allow identification of the relative metabolic activity of cortical neurons with or without compounds of formula (I).

FIG. 2A illustrates the results of a test using compounds of formula (I) on the activity of Neuro-2a cells under normal conditions (not deprived of oxygen and glucose), by measuring the optical density of the tetrazolium salt MTT. CTL means not treated (control). The results allow identification of the relative metabolic activity of Neuro-2a cells with or without compounds of formula (I).

FIG. 2B illustrates and demonstrates the beneficial effect of compounds according to formula (I) on the metabolic activity of Neuro-2a cells after ethanol (EtOH) intoxication.

FIG. 2C illustrates and demonstrates the beneficial effect of compounds according to formula (I) on mitochondrial activity in Neuro-2a cells after ethanol (EtOH) intoxication.

FIG. 3 illustrates the results of a test demonstrating the effect of compounds of formula (I) on the mitochondrial dynamics of a cell line.

FIG. 4 illustrates the results of a test demonstrating the inhibition of mitochondrial fission with compounds according to formula (I). A qRT-PCR analysis of cochlear explants treated with the compound DX243 enables observation of a reduction in drp1 mRNA after 24 h of DX243 treatment at 100 nM relative to untreated (NT) cochleae.

FIG. 5 illustrates the results of a test evaluating mitochondrial dynamics and mitochondrial stress in dopaminergic neurons.

FIG. 6 illustrates the results of a test evaluating mitochondrial stress in a culture of dopaminergic neurons.

FIG. 7 illustrates the results of a test evaluating mitochondrial stress in a culture of hippocampal neurons.

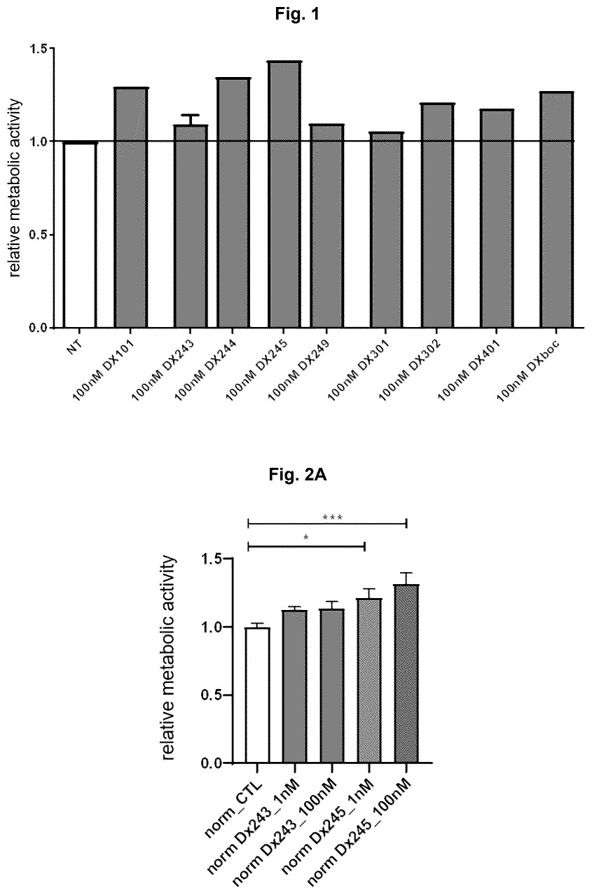

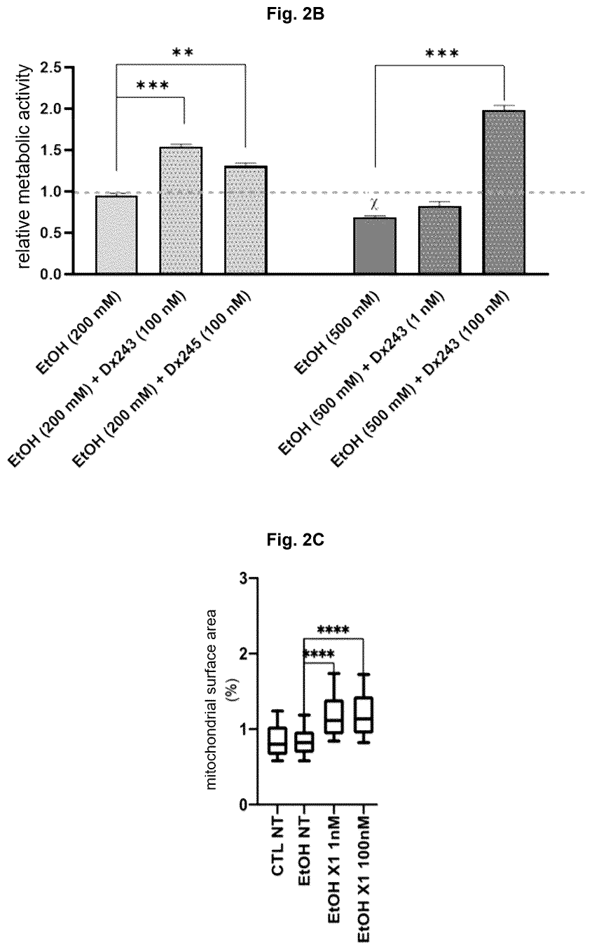

In FIGS. 2A, 2C, 3, 5 and 6, the stars indicate the statistical power of the results. One star indicates that it is 95% certain that the results are not due to chance. The presence of two stars indicates that it is 99% certain that the results are not due to chance, the presence of three stars indicates that it is 99.9% certain that the results are not due to chance, and the presence of four stars indicates that it is 99.99% certain that the results are not due to chance.

### DETAILED DESCRIPTION OF THE PREFERRED EMBODIMENTS

In this description, unless otherwise specified, it is understood that when a range is given, it includes the upper and lower limits of said range.

In the present invention, the following definitions apply:
“preventing a pathology” means avoiding the occurrence of diseases or injuries or maintaining and improving health; it may also be referred to as preventive treatment.
“ameliorating a pathology” means reducing the symptoms of said pathology; it may also be referred to as palliative treatment.
“treating a pathology” means curing a disease; it may also be referred to as curative treatment.
“mitochondrial deficiency” means a decrease in mitochondrial activity and/or mitochondrial dynamics and/or mitochondrial axonal transport, resulting in a loss of energy production and the accumulation of substances harmful to the body.
“mitochondrial activity” means the ability of mitochondria to regulate cellular metabolism and to produce energy (ATP) via their respiratory chain.
“mitochondrial dynamics” means the ability of mitochondria to fuse or fission, so as to preserve their morphology and size and/or to allow an increase in mitochondrial amount (mass) in cells.
“mitochondrial axonal transport” means the ability of mitochondria to move so as to meet the necessary energy demand at a precise point in the cell.
“mitochondrial fission” means the division of a mitochondrion into two distinct mitochondria.

In order to demonstrate that the compounds of formula (I) have an influence on the mitochondrial function of cells, various experiments were performed proving the beneficial effect of said compounds on mitochondrial function in general, through their effects on the number, dynamics, stress, transport or activity of mitochondria. Several experimental protocols are described hereinbelow which demonstrate the beneficial effect of the compounds of formula (I), notably those indicated in Table 2 below, under different conditions and experimental models.

TABLE 2
List of compounds of formula I used in the experimental examples.

| Name | Structure |
|---|---|
| DX101 | 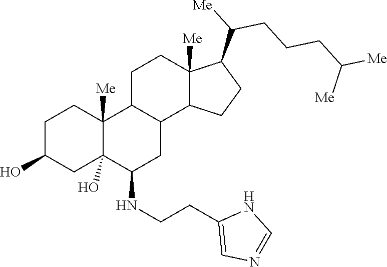 [SMILES: CC(C)CCCC(C)[C@H]1CCC2C3C[C@@H](NCCc4cnc[nH]4)[C@@]4(O)C[C@@H](O)CC[C@]4(C)C3CC[C@@]21C] |
| DX243 | 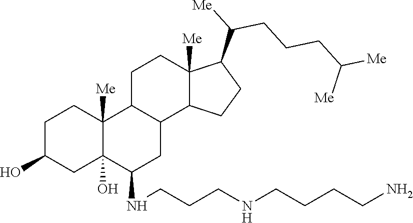 [SMILES: CC(C)CCCC(C)[C@H]1CCC2C3C[C@@H](NCCCNCCCCN)[C@@]4(O)C[C@@H](O)CC[C@]4(C)C3CC[C@@]21C] |
| DX242 | 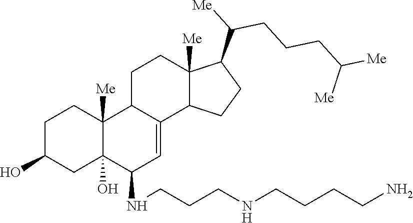 [SMILES: CC(C)CCCC(C)[C@H]1CCC2C3=C[C@@H](NCCCNCCCCN)[C@@]4(O)C[C@@H](O)CC[C@]4(C)C3CC[C@@]21C] |
| DX245 | 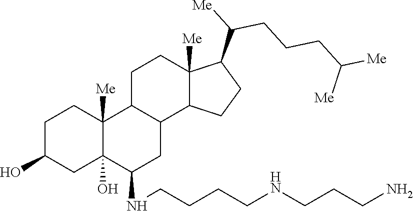 [SMILES: CC(C)CCCC(C)[C@H]1CCC2C3C[C@@H](NCCCCNCCCN)[C@@]4(O)C[C@@H](O)CC[C@]4(C)C3CC[C@@]21C] |
| DX244 | 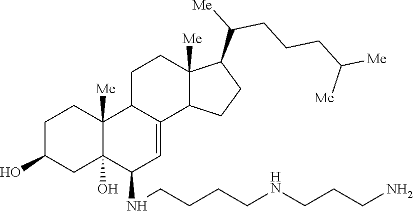 [SMILES: CC(C)CCCC(C)[C@H]1CCC2C3=C[C@@H](NCCCCNCCCN)[C@@]4(O)C[C@@H](O)CC[C@]4(C)C3CC[C@@]21C] |
| DX249 | 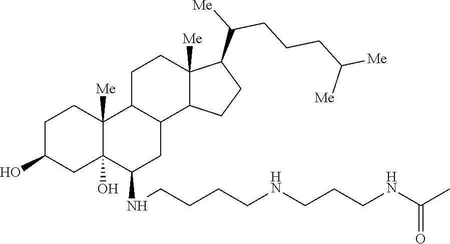 [SMILES: CC(=O)NCCCNCCCCN[C@@H]1CC2C3CC[C@H](C(C)CCCC(C)C)[C@@]3(C)CCC2[C@@]2(C)CC[C@H](O)C[C@]12O] |
| DX301 | 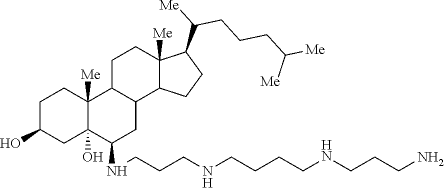 [SMILES: CC(C)CCCC(C)[C@H]1CCC2C3C[C@@H](NCCCNCCCCNCCCN)[C@@]4(O)C[C@@H](O)CC[C@]4(C)C3CC[C@@]21C] |
| DX302 | 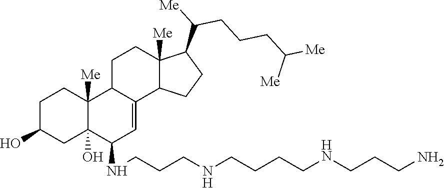 [SMILES: CC(C)CCCC(C)[C@H]1CCC2C3=C[C@@H](NCCCNCCCCNCCCN)[C@@]4(O)C[C@@H](O)CC[C@]4(C)C3CC[C@@]21C] |
| DX401 | 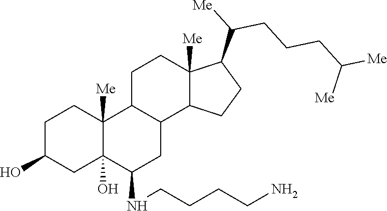 [SMILES: CC(C)CCCC(C)[C@H]1CCC2C3C[C@@H](NCCCCN)[C@@]4(O)C[C@@H](O)CC[C@]4(C)C3CC[C@@]21C] |
| DX243BOC | 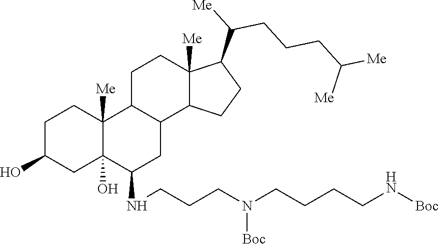 [SMILES: CC(C)CCCC(C)[C@H]1CCC2C3C[C@@H](NCCCN(CCCCNC(=O)OC(C)(C)C)C(=O)OC(C)(C)C)[C@@]4(O)C[C@@H](O)CC[C@]4(C)C3CC[C@@]21C] |

The concentrations or molarities of the compounds are expressed in moles per liter, the symbol for which is mol·L⁻¹ or M.

### Example 1: Test of Relative Metabolic Activity on Cortical Neurons

A protocol was developed for obtaining a primary culture of cortical neurons from cells collected from the brains of wild-type mouse embryos. Step 1 consisted in collecting embryonic cortexes to put cortical neurons under suitable culture conditions. The second step consisted in culturing the collected cells in Neurobasal™ Medium (Ref. 21103049 ThermoFisher Scientific) to which L-glutamine and B27 supplement 50× (Ref. 17504044 ThermoFisher Scientific) were added. Neurons from the primary culture were then isolated and purified. The abovementioned culture conditions afford a purified culture of neurons from the dissociation of the embryonic cortexes.

A test based on the metabolic activity of the neurons was performed to evaluate the effect of the compounds of formula (I) on this metabolic activity. This test is based on the use of the tetrazolium salt MTT (3-(4,5-dimethylthiazol-2-yl)-2,5-diphenyltetrazolium bromide). Tetrazolium is reduced by mitochondrial succinate dehydrogenase in active live cells to formazan, a purple-colored precipitate. The amount of precipitate formed is proportional to the metabolic activity present in the culture. Thus, spectroscopic assay of the optical density at 550 nm makes it possible to determine the relative amount of metabolic activity and consequently the mitochondrial activity. The results obtained are shown in FIG. 1. This test on cortical neuron cultures was performed with different compounds in reference to Table 2 (DX101, DX243, DX244, DX245, DX249, DX301, DX302 and DX401) used at the same concentration (100 nM).

With reference to FIG. 1, this test makes it possible to highlight a positive effect of the test compounds of formula (I) on mitochondrial activity relative to untreated neurons.

### Example 2: Test of Relative Metabolic Activity on Neuro-2a Neurons

The same test described in Example 1 was performed on Neuro-2a cells—a mouse neuroblastoma cell line. Under physiological conditions, once cultured and at about 50% confluence, Neuro-2a cells were placed in contact for 24 h with two concentrations of the compounds DX243 and DX245. An MTT assay performed following these 24 h of treatment made it possible to demonstrate that the compound DX243, but especially the compound DX245, allowed an increase in the metabolic activity of Neuro-2a cells, as represented in FIG. 2A.

### Example 3: Test of Metabolic Activity after Ethanol (EtOH) Intoxication

The same test described in Example 1 was performed on Neuro-2a cells—a mouse neuroblastoma cell line. It was shown that 24 h of intoxication with ethanol (EtOH) at high concentration (500 mM) in this cell line reduced the metabolic activity of these cells (χ=P value <0.0001 relative to the control). This metabolic activity was partially recovered with a 24 h treatment with 1 nM of compound DX243.

Advantageously, treatment for 24 h with 100 nM of compound DX243 or compound DX245 gives rise to significantly greater metabolic activity relative to the untreated cells, after intoxication with EtOH (200 mM and 500 mM), as illustrated in FIG. 2B.

Effects of the compound DX243 on mitochondria in Neuro-2a cells after ethanol intoxication are shown in FIG. 2C. This figure highlights the increase in mitochondrial surface area in the presence of the compound DX243, studied with the aid of a MitoTracker, after 24 h of ethanol intoxication (500 mm).

### Example 4: Test of Mitochondrial Dynamics on SHSY5Y and C2C12 Cell Lines

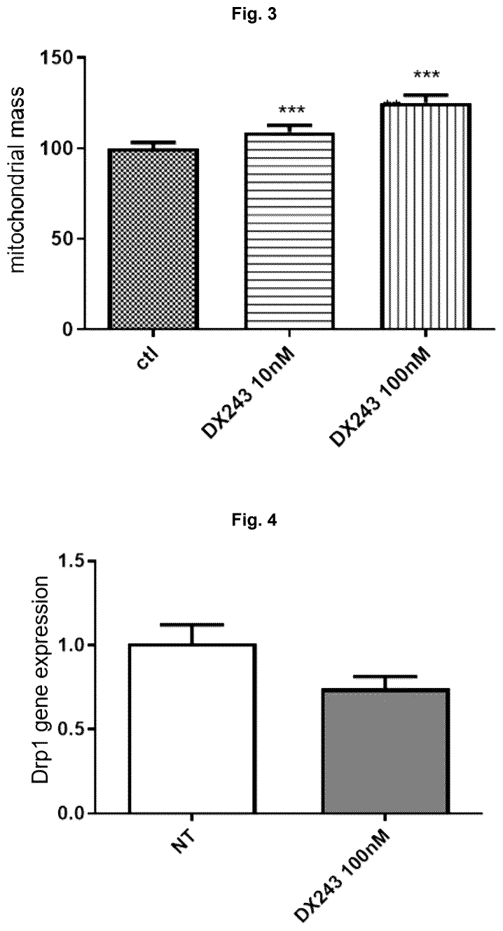

A human neuroblastoma cell line (SHSY5y) and also a myoblast cell line (C2C12) were cultured in DMEM™ medium (Ref. 21068028 ThermoFisher Scientific). Once in culture, these two cell lines were treated for 24 h with different concentrations of the compound DX243. A series of images was taken, labeling with Mitotracker™ (Ref. M7512, ThermoFisher Scientific), which highlights and visualizes the mitochondria present in these cells, and enables quantification of their amount. This quantification of mitochondrial mass revealed by means of Mitotracker labeling highlighted an effect of the compound DX243 on mitochondrial dynamics (increase in mitochondrial amount (mass)), as illustrated in FIG. 3.

### Example 5: Test to Evaluate Inhibition of Mitochondrial Fission on Cochlear Explants

A cochlear explant model was developed. To obtain cochleae, the organ of hearing, in culture, cochleae from 3-day-old mice were microdissected. These cochleae were then placed in DMEM medium with addition of glucose (0.5%), N1 (0.5%), insulin (0.25%) and penicillin (0.1%), for 24 h. 100 nM of the compound DX243 were then added to this culture medium for 24 h.

The cochleae were then recovered and the RNA extracted therefrom, so as to be able to analyze the modulation of several genes following treatment with the compound DX243. It was thus observed that treatment of cochlear explants with the compound DX243 (100 nM) entrained inhibition of expression of the mitochondrial fission gene drp1, as illustrated in FIG. 4. This test demonstrates the beneficial role of the compound DX243 on mitochondria, notably by inhibiting mitochondrial fission.

### Example 6: Test to Evaluate Mitochondrial Dynamics and Mitochondrial Stress in Dopaminerqic Neurons

The mitochondrial effects of compound DX243 were tested in a model of dopaminergic neurons treated with MPP+ (1-methyl-4-phenylpyridinium), a mitochondrial toxin that increases mitochondrial stress and reduces the number of mitochondria. MPP+ is known for its deleterious effects on mitochondria (increased mitochondrial stress measured by the amount of cytochrome C, reduced mitochondrial number measured by means of the Mitotracker test). Dopaminergic neurons were obtained from microdissection of the midbrain of rat embryos cultured with Neurobasal™ Medium (Ref. 21103049 ThermoFisher Scientific) supplemented with B27 supplement 50× (Ref. 17504044 ThermoFisher Scientific), penicillin/streptomycin, L-glutamine and also BDNF (brain-derived neurotrophic factor) and GDNF (glial cell-derived neurotrophic factor).

These neurons were cultured for 5 days and then treated for 48 h with MPP+. These neurons were also exposed to different concentrations of the compound DX243 during the 48 h of MPP+ treatment, and also 48 h after MPP+ removal.

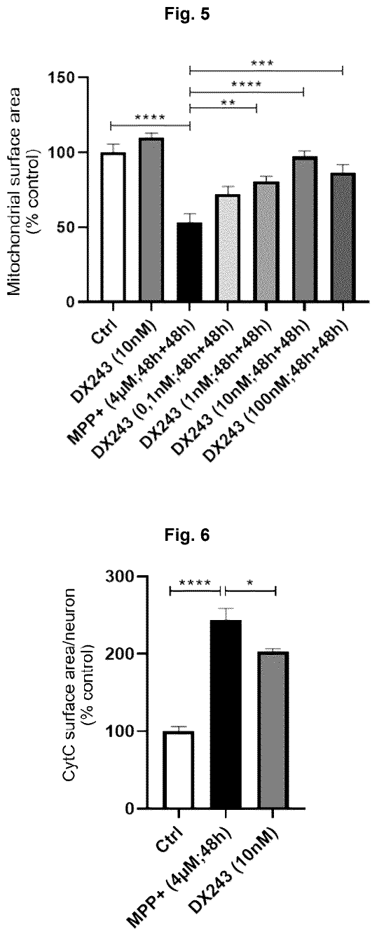

The results demonstrate that the compound DX243 is capable of increasing and restoring mitochondrial mass in these dopaminergic neurons which have lost part of this mass following MPP+ treatment, as illustrated in FIG. 5.

Furthermore, 10 nM of the compound DX243 also brings about a reduction in mitochondrial stress engendered by MPP+ treatment, as illustrated in FIG. 6.

### Example 7: Test to Evaluate Mitochondrial Dynamics and Mitochondrial Stress in Hippocampal Neurons

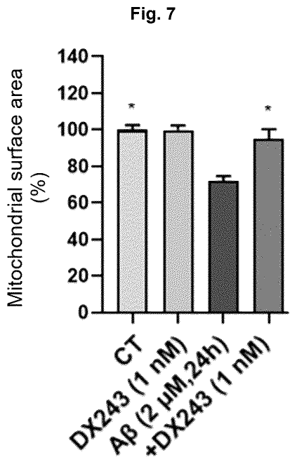

The mitochondrial effects of compound DX243 were tested in a model of hippocampal neurons treated with peptide Aβ 1-42, this peptide inducing mitochondrial stress (represented by a reduction in mitochondrial surface area). It was shown that the compound DX243 (1 nM) is capable of restoring this decrease in mitochondrial surface area, as illustrated in FIG. 7.

The use of the verb “to contain”, to comprise” or “to include” and its conjugated forms does not exclude the presence of elements or steps other than those stated in a claim.

In the claims, any reference sign in parentheses shall not be interpreted as a limitation of the claim.

1. A compound of formula (I): 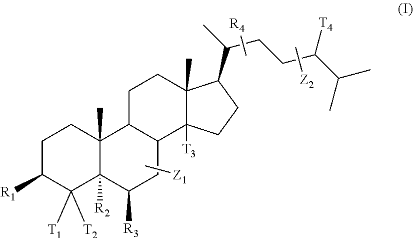
wherein
R₁=OH, F, OCₙH₂ₙ₊₁, OC(O)R, OC(O)OR, OC(O)NHR or OP(O)(OR)₂ with R=H or CₙH₂n+₁, with 1≤n≤8;
R₂=H or OH;
R₃=—NR₅R₆;
R₅ is H or —(CH₂)₃NH₂; and
R₆ is selected from the group consisting of —(CH₂)₃NR₇(CH₂)₄NHR₇, —(CH₂)₃NHR₇, —(CH₂)₄NHR₇, —(CH₂)₄N R₇(CH₂)₃NHR₇, —(CH₂)₃N R₇(CH₂)₄N R₇(CH₂)₃NHR₇, —(CH₂)₂-imidazol-4-yl, —(CH₂)₂-indol-3-yl, with R₇=H, C(O)OCH₃ or C(O)OC(CH₃)₃; and
R₄=H or OH in position 20, 22, 24, 25, 26 or 27, positioned to create an asymmetric center of R or S configuration;
Z₁ and Z₂ each represent the number of double bonds between carbon atoms C7 and C8 and C22 and C23 respectively (either 0 or 1); T₁, T₂ and T₃=H or CH₃ independently of each other; T₄=H, CH₃, C₂H₅ positioned to obtain an asymmetric center of R or S configuration in position 24;
for use in the treatment of a mitochondrial deficiency-related pathology selected from the group consisting of autism, spinal cord injury, multiple sclerosis, epilepsy, migraine, alcohol-related mental pathologies, ataxia, neuropathies, smoking-related cerebral and pulmonary disorders, MERRF syndrome and NARP syndrome.

2. The compound as claimed in claim 1, wherein the compound of formula (I) is defined by:
R₁=OH, F, OCₙH₂ₙ₊₁, OC(O)R, OC(O)OR, OC(O)NHR or OP(O)(OR)₂ with R=H or CₙH₂ₙ₊₁, with 1≤n≤8;
R₂=OH;
R₃=—NR₅R₆;
R₅=H;
R₆=—(CH₂)₃NR₇(CH₂)₄NHR₇, —(CH₂)₃NHR₇, —(CH₂)₄NHR₇, —(CH₂)₄N
R₇(CH₂)₃NHR₇, —(CH₂)₃N R₇(CH₂)₄N R₇(CH₂)₃NHR₇, —(CH₂)₂-imidazol-4-yl, —(CH₂)₂-indol-3-yl, with R₇=H, C(O)OCH₃ or C(O)OC(CH₃)₃;
Z₁=0or 1;
Z₂=0;
R₄=H.

3. The compound as claimed in claim 1, wherein the compound of formula (I) is defined by Z₁=0, Z₂=0; R₁=R₂=OH; R₄=H; R₅=H; T₁=T₂=T₃=T₄=H.

4. The compound as claimed in claim 3, wherein the compound of formula (I) is defined by R₆=—(CH₂)₄NH(CH₂)₃NHR₇ with R₇=C(O)CH₃ and is the compound 5α-hydroxy-6β-[3-(4-aminobutylacetamide)propylamino]cholestan-3β-ol.

5. The compound as claimed in claim 3 wherein the compound of formula (I) is defined by R₆=—(CH₂)₂NHR₇ with R₇=imidazol-4-yl and is 5α-hydroxy-63-[2-(1H-imidazol-4-yl)ethylamino]cholestan-3β-ol.

6. The compound as claimed in claim 3, wherein the compound of formula (I) is defined by R₆=—(CH₂)₃NR₇(CH₂)₄NHR₇, —(CH₂)₄NR₇ (CH₂)₃NHR₇, —(CH₂)₃NR₇(CH₂)₄NR₇(CH₂)₃NHR₇; or —(CH₂)₄NHR₇; and R₇=H and is, respectively, 5α-hydroxy-6β-[3-(4-aminobutylamino)propylamino]cholestan-3β-ol, 5α-hydroxy-6β-[4-(3-aminobutylamino)propylamino]cholestan-3β-ol, 5α-hydroxy-6β-{3-[4-(3-aminopropylamino)butylamino]propylamino}cholestan-3β-ol, 5α-hydroxy-6β-(4-aminobutylamino)cholestan-3β-ol.

7. The compound as claimed in claim 3, wherein the compound of formula (I) is defined by R₆=—(CH₂)₃NR₇(CH₂)₄NHR₇ and R₇=C(O)OC(CH₃)₃ and is 5α-hydroxy-6β-[3-(4-tert-butyloxycarbonylaminobutyl-tert- butyloxycarbonylamino)propylamino]cholestan-3β-ol.

8. The compound as claimed in claim 2, wherein the compound of formula (I) is defined by Z₁=1 and Z₂₌₀; R₁=R₂=OH; R₄=H; R₅=H; T₁=T₂=T₃=T₄=H.

9. The compound as claimed in claim 8, wherein the compound of formula (I) is defined by R₆=—(CH₂)₃NR₇(CH₂)₄NHR₇; —(CH₂)₄NR₇(CH₂)₃NHR₇ or —(CH₂)₃NR₇(CH₂)₄NR₇(CH₂)₃NHR₇; and R₇=H and is, respectively, Sa-hydroxy-6β-[3-(4-aminobutylamino)propylamino]cholest-7-en-3β-ol, 5α-hydroxy-6β-[4-(3-aminobutylamino)propylamino]cholest-7-en-3β-ol, 5α-hydroxy-6β-{3-[4-(3-aminopropylamino)butylamino]propylamino}cholest-7-en-3β-ol.

10. The compound as claimed in claim 1, wherein the mitochondrial deficiency-related pathology is due to a deficiency in mitochondrial dynamics and/or mitochondrial transport and/or mitochondrial activity.

11. A pharmaceutical composition comprising at least one of the compound of formula (I) as claimed in claim 1, a pharmaceutically acceptable salt of the compound of formula (I) as claimed in claim 1, or combinations thereof for use in treating a mitochondrial deficiency-related pathology selected from the group consisting of autism, spinal cord injury, multiple sclerosis, epilepsy, migraine, alcohol-related mental pathologies, ataxia, neuropathies, smoking-related cerebral and pulmonary disorders, MERRF syndrome and NARP syndrome.

12. The composition as claimed in claim 11, wherein the composition is in the form of an aqueous solution and has a concentration of compound of formula (I) of between 1 pmol·L⁻¹ and 1 mmol·L⁻¹.

13. The composition as claimed in claim 11, wherein the composition is in the form of an aqueous solution and has a concentration of compound of formula (I) of between 10 pmol·L⁻¹ and 0.1 mmol·L⁻¹.

14. The composition as claimed in claim 11, wherein the composition is in the form of an aqueous solution and has a concentration of compound of formula (I) of between 0.1 nmol·L⁻¹ and 1 pmol·L⁻¹.
